(19) United States
(12) Patent Application Publication
(10) Pub. No.: US 2007/0053980 A1
(43) Pub. Date: Mar. 8, 2007

(54) Extended release tiagabine formulations with reduced side-effects

(76) Inventors: Francisco Javier Alvarez, Wilmington, DE (US); Kathleen M. Apfelbaum, Lake Villa, IL (US); David M. Brown, Lake Villa, IL (US); Linda E. Gustavson, Evanston, IL (US); Russell T. Slade, Lindenhurst, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/592,694

(22) Filed: Nov. 3, 2006

Related U.S. Application Data
(63) Continuation of application No. 10/421,043, filed on Apr. 23, 2003.
Application No. 10/421,043 is a continuation of application No. 09/235,540, filed on Jan. 22, 1999, now abandoned.
(60) Provisional application No. 60/072,130, filed on Jan. 22, 1998.

(51) Int. Cl. A61K 9/22

(52) U.S. Cl. 424/468

(57) ABSTRACT

Extended release tiagabine formulations that demonstrate fewer side-effects when administered to a patient.

This application is a continuation of U.S. patent application Ser. No. 09/235,540, filed Jan. 22, 1999.

### FIELD OF THE INVENTION

Extended release formulations of tiagabine, an anti-epileptic drug, provides reduced side-effects and reduced titration times.

### BACKGROUND OF THE INVENTION

Tiagabine is used for controlling seizures in certain types of epilepsy. However, tiagabine sometimes produces uncomfortable side-effects, which if severe, may lead to discontinuation of anti-epileptic therapy with the compound. Certain of the side-effects are related to the central nervous system that are associated with reduced tolerance for the drug. Examples of such side effects include, ataxia, dizziness, headache, pharyngitis, and abnormal vision and thought processes.

To minimize adverse events from tiagabine therapy, one initiates tiagabine therapy by administering small doses, and then slowly and carefully increasing (titrating) the dosage to the optimal therapeutic level. This delays the time required to reach the therapeutically optimal tiagabine plasma concentration. The delay is detrimental not only because of the delay in controlling the seizures, but because side-effects may develop before the optimum treatment level is reached.

Extended release formulations of medicaments are well-known. However, such compositions have generally been utilized to prevent of deactivation of the drug in the intestinal tract before absorption into the blood stream, to maintain a more constant concentration of the drug in the blood, or to allow drug administration at less frequent intervals.

### SUMMARY OF THE INVENTION

Applicants have found that administering an extended release formulation of tiagabine to a patient produces fewer side-effects for the patient. As a further advantage, the extended release formulation also requires little or no titration phase, thus minimizing the time required for achieving seizure control. Furthermore, the extended release formulation may allow for a reduced frequency of dosing, e.g., once a day dosing.

Extended release compositions of tiagabine may be prepared in several forms, including matrix tablets and microparticulated pellets. Matrix tablets may contain hydrophilic polymers such as high molecular weight polyethylene oxide or hydroxypropylmethyl cellulose. Optional hydrophilic reagents may be added to modify the rate of release of the active ingredient.

Multiparticulate pellets of tiagabine may be prepared by encapsulating the drug with hydrophobic materials such as waxes, glyceryl behenate, triglycerides or mixtures of these materials. Again, optional hydrophilic reagents may be added to modify the rate of release of the active ingredient. An advantageous encapsulation process comprises suspending the drug in the molten material and forming small spherical particles as the molten material comes into contact with a disk rotating at high speed. The formed particles are cooled to solidify the hydrophobic encapsulating particles.

### BRIEF DESCRIPTION OF THE FIGURES

FIG. 1 shows the desired release profile of the extended release tiagabine hydrochloride tablet (12 mg, QD) as compared to the immediate release profile (4 mg, TID).

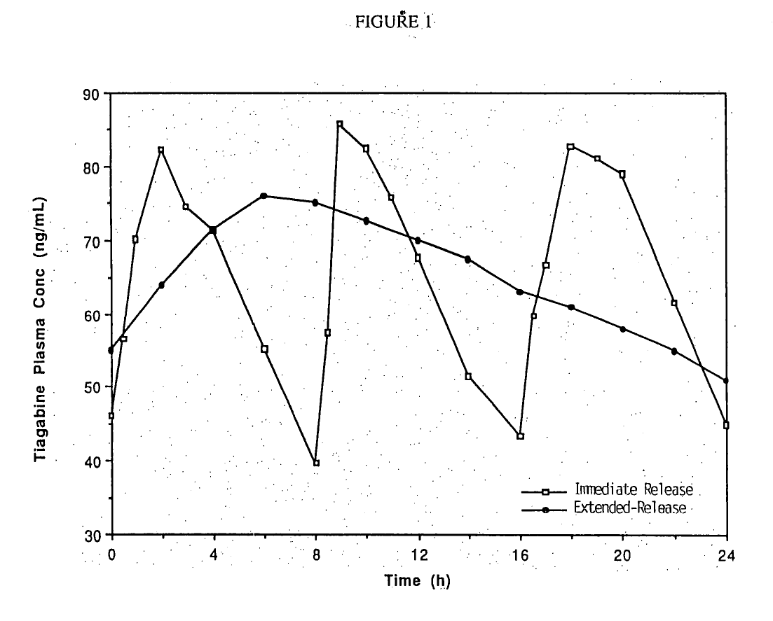

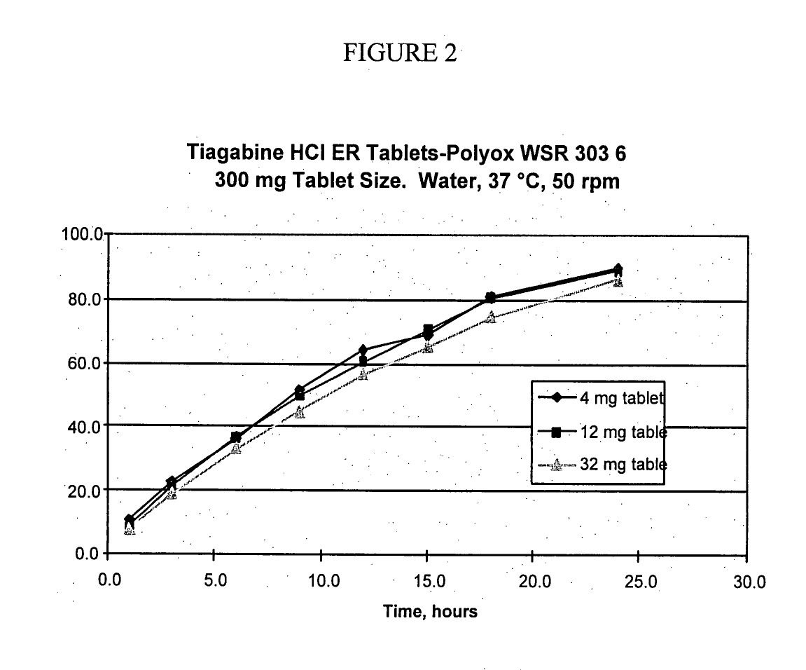

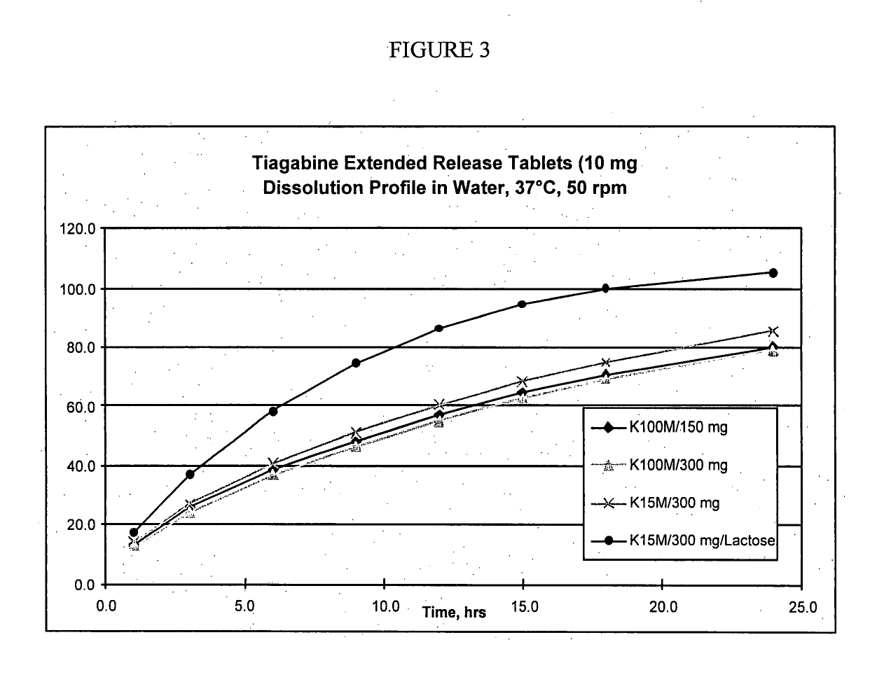

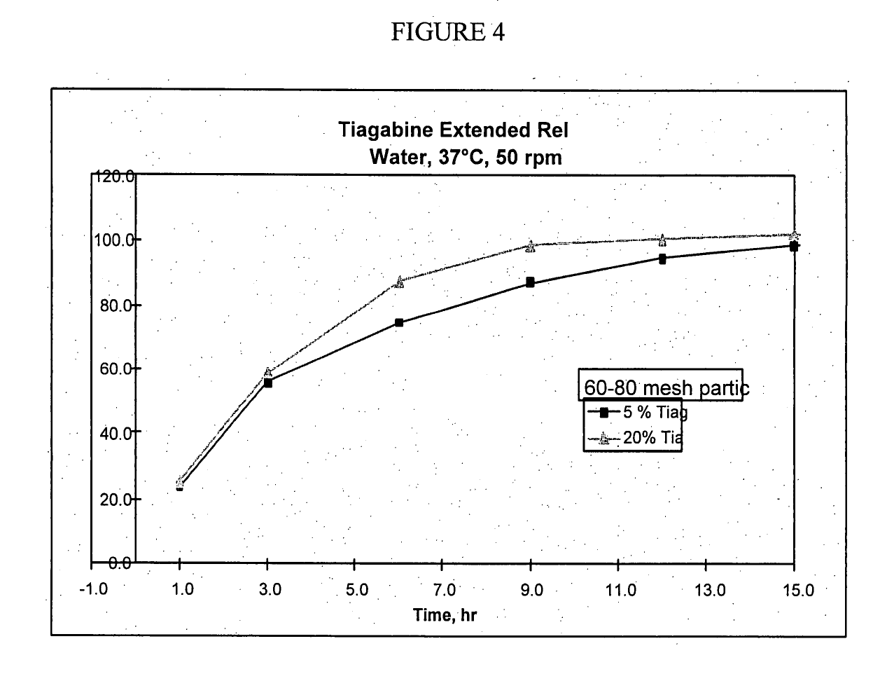

### DETAILED DESCRIPTION OF THE INVENTION

One embodiment of the invention is an extended-release composition comprising tiagabine combined in a matrix with a hydrophilic polymer such as high molecular weight polyethylene oxide (Polyox) or hydroxypropylmethyl cellulose (HPMC). One preferred polymer is Polyox. One preferred formulation is a tablet form. In an optional embodiment, other hydrophilic reagents, such as hydroxypropylmethyl cellulose or hydroxypropyl cellulose, for example, may be employed to modify the rate of release of the active ingredient.

Another embodiment of the invention is an extended release formulation comprising tiagabine encapsulated in the formulation with a hydrophobic material such as a wax, glyceryl behenate, triglycerides or mixtures of these materials. One preferred hydrophobic material is glyceryl behenate. A preferred formulation is a capsule form. In an optional embodiment, other hydrophilic reagents, such as hydroxypropyl methyl cellulose or hydroxypropyl cellulose, for example, may be employed to modify the rate of release of the active ingredient.

In a preferred embodiment, the extended release formulation comprises tiagabine in a tablet form in which the tiagabine is encapsulated in the formulation with glyceryl behenate by suspending the drug in the molten material and forming small spherical particles as the molten material comes into contact with a disk rotating at high speed, with subsequent cooling of the particles and subsequent formulation into capsules.

Process to Prepare Tablets

In order to prepare solid, shaped dosage forms from fine particles or powders comprising therapeutic agents, it is generally necessary to process the powders in a manner that improves flowability, cohesiveness and other characteristics which will enable the resulting material to be fabricated by conventional processes such as encapsulation, molding, tableting, etc. into a satisfactory unit form that can suitably deliver an agent to the patient.

Various processes have been developed for modifying starting powders or other particulate materials. Typically the powders are gathered together with a binder material into larger permanent free-flowing agglomerates or granules referred to collectively as a “granulation.” For example, solvent-assisted “wet” granulation processes are generally characterized in that the powders are combined with a binder material and moistened with water or an organic solvent under conditions to result in formation of a wet granulated mass from which the solvent must then be evaporated. Alternatively, known “dry granulation” processes can be used, depend on milling schemes, to produce a suitable granulation.

A “direct compression” process has, in limited cases, provided a simpler and more economical means of preparing compressed dosage forms. In such a process, the active ingredient is combined with a binder-diluent or vehicle which itself is characterized in having the requisite properties for tableting, such as flowability, appropriate particle size distribution, binding ability, acceptable bulk and tap density and dissolution properties, so that the resulting blend can be “directly” provided to a die cavity or mold for compaction, without prior granulation. See U.S. Pat. No. 5,273,758 to Shangraw; “Compressed Tablets by Direct Compression,” Pharmaceutical Dosage Forms 2d Ed., v. 1, pp. 195-246 (1989).

A suitable direct compression vehicle f6r a given application is preferably also tailored, for example, to be compatible with the active ingredient; to resist physical or chemical change on aging; to be air, moisture and heat-stable; have sufficient capacity for the active ingredient in the dosage form; to accept colorants uniformly when necessary; and not to interfere with biological availability.

Materials employed by the art which to varying degrees fulfill the requirements of a direct compression vehicle include water soluble materials such as various forms of lactose (e.g., spray-dried lactose, Fast Flow″ lactose, anhydrous lactose), as well as sucrose, dextrose, sorbitol, mannitol and maltodextrin, and relatively insoluble materials such as microcrystalline cellulose (e.g., Avicel″), starch, dicalcium phosphate dihydrate, and calcium carbonate.

However, such materials, while often comprising a relatively large proportion by weight of the tableted formulation in order to impart full advantage of their compression properties, nevertheless in themselves are generally insufficient to regulate the rate of disintegration of the dosage form or release of the medicament, and therefore must often be accompanied by various additional excipients having such a rate-control effect, the latter which (given practical limitations on the size of the dosage form) may be confined to low concentrations at which the rate control effect is not completely satisfactory.

Polyox is a nonionic homopolymer of the formula —(—O—CH2-CH2-)ₙ-, wherein n represents the average number of oxyethylene groups, n generally being from about 2,000 to about 100,000. It is a water soluble resin which is available as a white powder in several grades which vary in viscosity profile when dissolved in water. National Formulary XVII, pp. 1963-1964 (1990). Molecular weights range from about 100,000 to about 8,000,000, corresponding to a viscosity range of under about 200 cps for a 5% aqueous solution of the lower molecular weight polymers to about 7,000 to 10,000 cps for a 1% solution of the higher molecular weight polymers. Polyethylene oxide resins are commercially available under the tradename Polyox® from Union Carbide Corporation. Polyox® WSR 303 has an average molecular weight of about 7,000,000, and a 1% aqueous solution thereof at 25° C. has a viscosity of about 7,200 to 10,000 cps on a Brookfield RVF, No. 2 spindle at 2 rpm, and a pH of 8 to 10.

The use of a particular molecular weight polyethylene oxide polymer as a binder material will depend on the desired disintegration or release rate characteristics to be imparted to the prepared dosage form. In general, lower molecular weight polyethylene oxide polymers, i.e. having MW of up to about 300,000, e.g., Polyox® N80, may be selected to prepare tablets from which the medicament is released within a relatively short time period. Sustained release dosage forms may be prepared from the higher molecular weight polymers, i.e. having MW higher than about 300,000, especially about 2,000,000 to 7,000,000 (e.g., Polyox® 303 and Polyox® WSR Coagulant). It is contemplated that mixtures of varying molecular weight polymers may also be employed as a matrix system to obtain the desired tablet release properties, and such mixtures may comprise respective amounts of the various polyethylene oxide polymers as shall be within the skill of the worker in the art to ascertain to provide the appropriate release pattern.

Other optional components of the compositions of the invention include various fillers, binders, disintegrants, diluents, hydrophilic polymers, etc., including cellulose ethers, such as HPMC, and waxy substances, as well as minor amounts of various lubricants such as talc, colloidal silicon dioxide, stearic acid or metal stearates, etc., and colorants, sweeteners, antioxidants, and the like.

Suitable fillers include microcrystalline cellulose, starch and sugars such as lactose and mannitol, which may be employed in amounts from about 5% to about 80% of the blend. Higher molecular weight polymers, for example, Polyox® 303 and Polyox® WSR Coagulant, may be employed in amounts from about 15% to about 80% of the blend. Hydrophilic polymers, such as hydroxypropyl methyl cellulose or hydroxypropyl cellulose, for example, may be employed in amounts from about 28% to 60% of the blend. Silicon dioxide may be employed in amounts from about 0.1% to about 4% of the blend. Other lubricants, such as waxes, hydrogenated vegetable oil, stearic acid, calcium stearate, magnesium stearate, mineral oil, and talc, for example, may be employed in amounts from about 0.05% to about 6% of the blend. Antioxidants, such as Vitamin E, BHA, and BHT, for example, may be employed in amounts from about 0.1% to about 1.5% of the blend.

Tiagabine, the active ingredient of the invention comprises from about 0.01 to about 95 wt. % of such compositions. A preferred composition of the invention comprises from about 0.5% to about 30 wt. % of tiagabine and about 15 to 80 wt. %, of free-flowing, directly compressible polyethylene oxide binder material.

The dosage forms may be prepared by a direct compression process; that is, the process consists essentially of, the steps of (i) dry blending particles comprising 15 to 80 wt. %, and preferably 20 to 60 wt. %, of polyethylene oxide with about 0.5 to about 30% of tiagabine, the therapeutic medicament, as well as other optional excipients, and (ii) providing the resulting mixture to a compression machine, and applying sufficient pressure to the composition to form a unitary dosage form.

The medicament may be employed in powder, crystalline, or other form, and typically need not be compounded to an amorphous or other type granulated form.

In one embodiment, the polyethylene oxide and medicament and other optional ingredients are dry blended, i.e. in the absence of added solvents or heat, to produce a free-flowing material wherein the medicament is well dispersed in the polyethylene binder-matrix. The mixture is then provided to, for example, a tableting machine and a compression force of about 0.5 to 10 tons is applied to prepare a tablet dosage form. In such a tablet, the medicament is generally evenly dispersed throughout the polyethylene oxide binder, and which is free of solvent residues.

As used herein, the term “tablet” refers to a compressed body which is composed of a plurality of discrete particles, and includes pills, lozenges, dragee cores, capsule slugs, molded forms, and the like.

In another embodiment, a hydrophobic ingredient, such as glyceryl behenate, beeswax, carnauba wax, triglycerides, and hydrogenated vegetable oils, and medicament and other optional ingredients are blended in a heated mixer. The molten material is then dropped at a rate of about 30 mL/min to about 300 mL/min, and preferably about 100 mL/min, onto a disk rotating from about 1000 to 5000 rpm, and preferably about 3000 rpm. The droplets thrown off the disk are solidified by air-cooling and collected. In a preferred embodiment, glyceryl behenate is the hydrophobic ingredient. These particles are then placed into a gelatin capsule to form a capsule dosage form.

As used herein, the term “capsule” refers to a gelatin capsule which is filled with a plurality of discrete particles formed from a molten blend of tiagabine plus a hydrophobic ingredient and other optional ingredients.

The expression “therapeutic medicament” or “drug” shall include any physiologically or pharmacologically active substance that produces a local or systemic effect(s) in animals, which include warm-blooded mammals, humans, primates, etc.

The term “physiological” as used herein denotes the administration of a drug to effect normal levels and functions. The term “pharmacological” denotes variations in response to the amount of drug administered to the host. The devices have found a particular use as vehicles for various human and animal drugs, particularly for the oral administration thereof, although for other systems as well, including systems such as buccal, implant, nose, artificial gland, rectum, cervical, intrauterine, occular, arterial, venous, ear and the like, may be manufactured according to the process of the invention.

Compositions according to the invention may comprise tiagabine in free base form, or in a pharmaceutically acceptable salt form, preferably HCl. The chemical name for tiagabine is (−)-(R)-1-[4,4-bis(3-methyl-2-thienyl)-3-butenyl]nipecotic acid. Unless otherwise expressly indicated the chemical substances used are in the National Formulary or the U.S. Pharmacopeia.

### EXAMPLES OF FORMULATIONS

### Example 1

### Matrix Formulations Comprising (Polyox)

Ingredients shown in the table below were mixed in a V-blender for about 5-30 minutes. The powder was then compressed into 300 mg tablets by means of a tablet press, applying about 500 to 3500 pounds of compression force.

Four different blends were prepared, as shown, each having a different amount of tiagabine per table. The A4; A12, A20 and A32 tablets contained 4, 12, 20 and 32 mg of tiagabine per tablet, respectively.

| | | % of Formula | | | |
|---|---|---|---|---|---|
| Item | Name | A4 | A12 | A20 | A32 |
| 1 | Microcrystalline cellulose | 32.1 | 28.7 | 25.3 | 20.2 |
| | (Avicel PH 102) | | | | |
| 2 | Colloidal Silicon Dioxide, NF | 0.8 | 1.4 | 2.0 | 2.9 |
| | (Cab-O-Sil M5) | | | | |
| 3 | Vitamin E (dl-alpha tocopherol), USP | 0.5 | 0.5 | 0.5 | 0.5 |
| 4 | Water, Purified, USP (distilled) | qs | qs | qs | qs |
| 5 | Tiagabine Hydrochloride | 1.4 | 4.2 | 7.0 | 11.2 |
| 6 | Polyox WSR Coagulant (MW 5 MM) | 60.0 | 60.0 | 60.0 | 60.0 |
| 7 | Wax, Hydrogenated Vegetable Oil | 5.0 | 5.0 | 5.0 | 5.0 |
| | (Sterotex K) | | | | |
| 8 | Magnesium Stearate, NF, | 0.2 | 0.2 | 0.2 | 0.2 |
| | Impalpable powder | | | | |

### Example 2

### Matrix Formulations Comprising HPMC

Ingredients shown in the table below were mixed in a V-blender for 5-30 minutes. The powder was then compressed into 300 mg tablets by means of a tablet press, applying about 500 to 3500 pounds of compression force.

Two different blends were prepared, as shown, each having a different amount of tiagabine per table. The F1 and F2 tablets contained 28 and 14 mg of tiagabine per tablet, respectively.

| | | % per Tablet | |
|---|---|---|---|
| Item | Name | F-1 | F-2 |
| 1 | Microcrystalline cellulose (Avicel PH 102) | 54.7 | 59.4 |
| 2 | Colloidal Silicon Dioxide, NF (Cab-O-Sil M5) | 0.5 | 0.5 |
| 3 | dl-alpha tocopherol, Vitamin E | 0.5 | 0.5 |
| 4 | Tiagabine HCl Tablet Pre-Mix* | 9.3 | 4.7 |
| 5 | Hydroxypropyl Methyl Cellulose | 30.0 | 30.0 |
| | (K15M or K100M) | | |
| 6 | Wax, Hydrogenated Veg. Oil (Sterotex K) | 5.0 | 5.0 |

*Premix contained 82% tiagabine HCl and 18% SiO₂

### Example 3

### Additional Matrix Formulations Comprising Polyox

Ingredients shown in the table below were mixed in a V-blender for 5-30 minutes. The powder was then compressed into 300 mg tablets by means of a tablet press, applying about 500 to 3500 pounds of compression force.

Three different blends were prepared, as shown, each having a different amount of tiagabine per blend. Tablets were prepared from each blend, and each table contained 10 mg of tiagabine per tablet.

The tablets from this example were used in the in vivo study described in Example 6 below.

| | | % per Tablet | | |
|---|---|---|---|---|
| Item | Name | lot-172 | lot-173 | lot-174 |
| 1 | Cellulose (Avicel PH 102) | 70.7 | 25.7 | 30.3 |
| 2 | Tiagabine HCl Tablet Pre-Mix* | 9.4 | 9.4 | 4.7 |
| 3 | Polyethylene Oxide (Polyox WSR | 15.0 | 60.0 | 60.0 |
| | 303) | | | |
| 4 | Wax, Hydrogenated Veg. Oil | 5.0 | 5.0 | 5.0 |
| | (Sterotex K) | 100.0 | 100.0 | 100.0 |

*Premix contained 82% tiagabine HCl and 18% SiO₂

### Example 4

### Multiparticulate Formulations Comprising Hydrophobic Excipients

Ingredients shown in the table below were blended in a heated mixer, and the molten material was dropped and the rate of 100 mL per minute onto a disk rotating at 3000 rmp. The droplets thrown off the disk were air cooled, and the solidified particles were collected.

Two different blends were prepared, as shown, each having a different amount of tiagabine per unit weight of particulate material. The F3 and F4 tablets contained 5 and 21 mg of tiagabine, respectively, per 100 mg of particulate material.

% per Tablet

| Item | Name | F-3 | F-4 |
|---|---|---|---|
| 1 | Glyceryl Behenate, Compritol 888 ATO | 93.1 | 74.0 |
| 2 | d-alpha tocopherol (Vitamin E) | 0.5 | 0.5 |
| 3 | Tiagabine HCl Tablet Pre-Mix | 6.4 | 25.5 |

### Example 5

### Dissolution Profiles of Various Tiagabine Formulations

Formulations of reference immediate release formulations and the experimental formulations described prepared as described in Examples 1-3 above were tested in a multiple position dissolution stirrer such as that described at USP p. 1244, which was equipped with a Teflon paddle (50 rpm) in each of six vessels. A dissolution medium comprising 900 mL. of deaerated and distilled water was maintained at 37 ° C.±0.5° C. A tablet was sequentially dropped into each vessel. Stirring and timing (time zero) was commenced as the first tablet hit the bottom of the vessel (under the paddle).

At regular intervals, aliquots of test solution were withdrawn from each of the vessels in the order in which the tablets were originally dropped, using a stainless steel cannula. The aliquots were withdrawn from a point midway between the surface of the dissolution medium and the top of the paddle and not less than 1 cm. from each vessel wall. The amount of tiagabine present in each of the vessels was calculated by reference to standard solutions using HPLC. Dissolution profiles of the formulations from Examples 1-3 above are shown in Graphs 1-3 below.

Graph 1 shows the cumulative amount of tiagabine dispensed over a prolonged period of time from an extended release matrix formulation using high molecular weight polyethylene oxide.

[image]

Graph 2 shows the cumulative amount of tiagabine dispensed over a prolonged period of time from an extended release matrix formulation using hydroxypropylmethyl cellulose.

[image]

Graph 3 shows the cumulative amount of tiagabine dispensed over a prolonged period of time from an extended release formulation in a multiparticulate system.

[image]

As Graphs 1-3 demonstrate, no more than 80% of the tiagabine was released in less than 5 hours. By contrast, a standard immediate release formulation of tiagabine (Gabitril™, obtained from Novo-Nordisk) was found to release greater than 80% of the drug within 60 minutes or less.

### Example 6

### In Vivo Comparison Between Extended Release Tiagabine and Immediate Release Formulation

Several formulations containing high molecular weight polyethylene oxide were compared in a human bioavailability study where it was found that they were bioequivalent to the immediate release dosage form. One Tiagabine extended release tablet containing 10 mg was administered orally to 16 fasting healthy male subjects. Of these, 13 completed the study. A summary of the phamacokinetic results (mean±SD) is presented in the Table below. The pharmacokinetic profiles showed a significant decrease in Cₘₐₓ and an increase in tₘₐₓ for the extended release formulation.

Pharmacokinetic Profiles of Extended Release Formulations
Compared with Gabitril ™ Control

| | Cₘₐₓ | Tₘₐₓ | AUC_{0-∞} | t_{1/2} |
|---|---|---|---|---|
| Regimen | (mg/mL) | (hr) | (mg hr/mL | (hr)† |
| Lot 172 | 70.8 ± 10.8* | 3.7 ± 0.6* | 1283 ± 279 | 9.1 |
| Lot 173 | 60.7 ± 10.6* | 6.8 ± 5.3* | 1266 ± 268** | 9.7 |
| Lot 174 | 54.8 ± 8.8* | 8.1 ± 3.5* | 1320 ± 272 | 11.2 |
| Gabitril control | 241.3 ± 59.1 | 0.7 ± 0.2 | 1387 ± 277 | 9.5 |

†Harmonic mean.
*Statistically significant difference as compared to control (p ≧ 0.05).
**Statistically significant difference as compared to control (p ≧ 0.05) for analysis of log-transformed AUC_{0-∞} only.

However, and unexpectedly, the number of adverse events observed during the clinical study, particularly those related to the central nervous system, were fewer than those observe with the immediate release formulation (see Table below). The side effects profile observed with the immediate release tiagabine formulation are consistent with those observed with these type of formulations in other clinical studies. The data indicate that an extended release formulation can provide comparable therapy with fewer adverse events.

Adverse Events Occurring in Two or More Subjects for
Any One Regimen of Tiagabine
Extended Release Formulation: Tiagabine Tablet,
10 mg

| | lot-172 | lot-173 | lot-174 | Reference |
|---|---|---|---|---|
| Adverse Event | N = 15 | N = 15 | N = 14 | N = 15 |
| Ataxia | 0 | 0 | 0 | 2 (13.3%) |
| Dizziness | 2 (13.3%) | 0 | 0 | 11 (73.3%) |
| Headache | 2 (13.3%) | 3 (20.0%) | 2 (14.3%) | 1 (6.7%) |
| Thinking | 0 | 0 | 0 | 5 (33.3%) |
| Abnormal | | | | |
| Pharyngitis¹ | 2 (13.3%) | 1 (6.7%) | 0 | 1 (6.7%) |
| Abnormal Vision | 0 | 0 | 0 | 2 (13.3%) |

¹In each case, the event was considered to have no relationship to tiagabine

### Example 7

### Additional In Vivo Comparison Between Extended Release Tiagabine and Immediate Release Formulation

A 12 milligram tiagabine hydrochloride extended release tablet, Formulation A12 in Example 1, was compared to a 4 milligram immediate release tablet. The extended release tablet was administered each morning for five (5) days under fasting conditions while the immediate release tablet was administered every eight (8) hours for five (5) consecutive days under nonfasting conditions.

A total of fourteen healthy subjects were tested in two stages. In the first stage, seven subjects received the extended release tablet and seven subjects received the immediate release tablet for the five days. A washout interval of seven days separated the last dose of stage 1 from the first dose of stage 2. In stage 2, the seven subjects that received the extended release tablet in stage 1 now received the immediate release tablet and the seven subjects who initially received the immediate release tablet now received the extended release tablet.

Blood samples were collected for determination of plasma tiagabine concentrations at 0 hours, 0.5, 1, 2, 3, 4, 6, 8, 8.5, 9, 10, 11, 12, 14, 16, 16.5, 17, 18, 19, 20, 22, and 24 hours after the morning dose on Day5 in each period. Additional blood samples were collected before the morning dose on Days 1, 3, and 4 in each period.

Blood samples were placed in an ice bath and protected from light. Plasma was separated from whole blood within one hour of collection by refrigerated centrifugation and stored frozen (≦20° C.).

Plasma concentrations of tiagabine were determined using a validated liquid chromatography method with tandem mass spectrometric detection (LC/MS/MS). A monomethyl analog of tiagabine was used as an internal standard.

A summary of the pharmacokinetic results (mean±SD) is presented in the Table below. The pharmacokinetic profiles showed a significant decrease in Cₘₐₓ and an increase in tₘₐₓ for the extended release tablet.

Pharmacokinetic Profiles of Extended Release Formulations
Compared with Gabitril ™ Control

| | Cₘₐₓ | Tₘₐₓ | AUC₀₋₂₄ | Cmin |
|---|---|---|---|---|
| Regimen | (ng/mL) | (hr) | (ng hr/mL | (ng/mL) |
| Extended | 84.0 ± 26.1* | 4.7 ± 5.2* | 1354 ± 551 | 34.5 ± 21.3 |
| Release | | | | |
| Immediate | 105.3 ± 17.0 | 1.6 ± 1.1* | 1573 ± 311 | 37.4 ± 11.0 |
| Release | | | | |

*Statistically significant difference as compared to immediate release tablet (p = 0.0069).

The present invention relates to extended release formulations of tiagabine and its salts. In particular, extended release formulation of tiagabine hydrochloride are provided for oral administration to mammals, and in particular, human patients. Preferred tiagabine formulations provide an extended-release oral administration to human patients, comprising from about 4 to about 80 mg tiagabine or a salt thereof, said formulation providing a mean maximum plasma concentration of tiagabine from about 10 to 1000 ng/mL from a mean of about. 2 to 8 hours after administration, and a mean minimum plasma concentration of tiagabine from about 1 to 700 ng/mL from a mean of about 22 to 26 hours after repeated administration every 24 hours through steady-state conditions.

An extended release formulation showing the release profile above comprising tiagabine may be combined in a matrix with a hydrophilic polymer selected from the group consisting of high molecular weight polyethylene oxide and hydroxypropylmethyl cellulose.

An extended release formulation comprising tiagabine may be encapsulated in a formulation with a hydrophobic material selected from the group consisting of waxes, glyceryl behenate, triglycerides and mixtures thereof.

Another preferred embodiment of the present invention includes an extended-release oral tiagabine tablet, comprising from about 4 to about 80 mg tiagabine or a salt thereof, said tablet providing a mean maximum plasma concentration of tiagabine from about 10 to 1000 ng/mL from a mean of about 2 to 8 hours after administration, and a mean minimum, plasma concentration of tiagabine from about 1 to 700 ng/mL from a mean of about 22 to 26 hours after repeated administration every 24 hours through steady-state conditions.

An extended release tablet comprising tiagabine may be combined in a matrix with a hydrophilic polymer selected from the group consisting of high molecular weight polyethylene oxide and hydroxypropylmethyl cellulose.

An extended release tablet comprising tiagabine may be encapsulated in a formulation with a hydrophobic material selected from the group consisting of waxes, glyceryl behenate, triglycerides and mixtures thereof.

We claim:

1. An extended release formulation comprising tiagabine combined in a matrix with a hydrophilic polymer selected from the group consisting of high molecular weight polyethylene oxide and hydroxypropylmethyl cellulose.

2. The formulation according to claim 1 wherein the hydrophilic polymer is selected from high molecular weight polyethylene oxide.

3. The formulation according to claim 2 wherein the formulation is shaped in a tablet form.

4. The formulation according to claim 1 wherein the hydrophilic polymer is hydroxypropylmethyl cellulose.

5. The formulation according to claim 4 is shaped in a in a tablet form.

6. An extended release formulation comprising tiagabine encapsulated in a formulation with a hydrophobic material selected from the group consisting of waxes, glyceryl behenate, triglycerides and mixtures thereof.

7. The formulation according to claim 6 wherein the hydrophobic material is glyceryl behenate.

8. The formulation according to claim 7 wherein the hydrophobic material selected from glyceryl behenate in a capsule form in which the tiagabine is encapsulated in the formulation with glyceryl behenate by suspending the drug in the-molten material and forming small spherical particles as the molten material comes into contact with a disk rotating at high speed, with subsequent cooling of the particles and subsequent formulation into capsules.

9. An extended release formulation comprising about 0.5 to 30 wt. % tiagabine combined in a matrix having:
a. about 15 to 80 wt. % of high molecular weight polyethylene oxide;
b. about 5 to 80 wt. % of a filler selected from the group consisting of microcrystalline cellulose, starch, and sugars;
c. about 0.1 to 4 wt. % of silicon dioxide;
d. about 0.1 to 1.5 wt. % of a preservative selected from the group consisting of tocopherol, BHA and BHT;
e. about 0.1 to 6 wt. % of a first lubricant selected from the group consisting of a wax, stearic acid, mineral oil, stearate and a hydrogenated vegetable oil; and
f. about 0.05 to 1 wt. % of a second lubricant selected from the group consisting of magnesium stearate, calcium stearate and talc.

10. An extended release formulation comprising about 0.5 to 30 wt. % tiagabine combined in a matrix having:
a. about 28 to 60 wt. % of a hydrophilic polymer selected from the group consisting of HPMC and hydroxypropyl cellulose;
b. about 5 to 80 wt. % of a filler selected from the group consisting of microcrystalline cellulose, starch, and sugars;
c. about 0.1 to 4 wt. % of silicon dioxide;
d. about 0.1 to 1.5 wt. % of a preservative selected from the group consisting of tocopherol, BHA and BHT;
e. about 0.1 to 6 wt. % of a first lubricant selected from the group consisting of a wax, stearic acid, mineral oil, stearate and a hydrogenated vegetable oil; and
f. about 0.05 to 1 wt. % of a second lubricant selected from the group consisting of magnesium stearate, calcium stearate and talc.

11. An extended release formulation comprising about 0.5 to 30 wt. % tiagabine encapsulated in a formulation having:
a. about 50 to 99.5 wt. % of a hydrophobic ingredient selected from the group consisting of glyceryl behenate, bees wax carnauba wax, triglycerides and hydrogenated vegetable oil;
b. about 0.1 to 4 wt. % of silicon dioxide; and
d. about 0.1 to 1.5 wt. % of a preservative selected from the group consisting of tocopherol, BHA and BHT.

12. An extended-release tiagabine formulation, comprising from about 4 to about 80 mg tiagabine or a salt thereof, said formulation providing a mean maximum plasma concentration of tiagabine from about 10 to 1000 ng/mL from a mean of about 2 to 8 hours after administration, and a mean minimum plasma concentration of tiagabine from about 1 to 700 ng/mL from a mean of about 22 to 26 hours after repeated administration every 24 hours through steady-state conditions.

13. An extended release formulation of claim 12 comprising tiagabine combined in a matrix with a hydrophilic polymer selected from the group consisting of high molecular weight polyethylene oxide and hydroxypropylmethyl cellulose.

14. The formulation according to claim 13 wherein the hydrophilic polymer is high molecular weight polyethylene oxide.

15. The formulation according to claim 13 wherein the formulation is shaped in a tablet form.

16. The formulation according to claim 13 wherein the hydrophilic polymer is hydroxypropylmethyl cellulose.

17. An extended release formulation of claim 12 comprising tiagabine encapsulated in a formulation with a hydrophobic material selected from the group consisting of waxes, glyceryl behenate, triglycerides and mixtures thereof.

18. The formulation according to claim 17 wherein the hydrophobic material is glyceryl behenate.

19. An extended-release oral tiagabine tablet, comprising from about 4 to about 80 mg tiagabine or a salt thereof, said tablet providing a mean maximum plasma concentration of tiagabine from about 10 to 1000 ng/mL from a mean of about 2 to 8 hours after administration, and a mean minimum plasma concentration of tiagabine from about 1 to 700 ng/mL from a mean of about 22 to 26 hours after repeated administration every 24 hours through steady-state conditions.

20. An extended release tablet of claim 19 comprising tiagabine combined in a matrix with a hydrophilic polymer selected from the group consisting of high molecular weight polyethylene oxide and hydroxypropylmethyl cellulose.

21. The extended release tablet according to claim 20 wherein the hydrophilic polymer is high molecular weight polyethylene oxide.

22. The extended release tablet according to claim 20 wherein the hydrophilic polymer is hydroxypropylmethyl cellulose.

23. An extended release tablet of claim 19 comprising tiagabine encapsulated in a formulation with a hydrophobic material selected from the group consisting of waxes, glyceryl behenate, triglycerides and mixtures thereof.

24. The formulation according to claim 23 wherein the hydrophobic material is glyceryl behenate.
